(19) United States
(12) Patent Application Publication
(10) Pub. No.: US 2005/0209686 A1
(43) Pub. Date: Sep. 22, 2005

(54) Blood vessel holding and positioning system

(75) Inventors: Gary W. Guenst, Collegeville, PA (US); Christopher Olig, Eden Prairie, MN (US)

Correspondence Address: FOLEY & LARDNER, 777 EAST WISCONSIN AVENUE, SUITE 3800, MILWAUKEE, WI, 53202-5308, US

(73) Assignee: Medtronic, Inc.

(21) Appl. No.: 10/804,391

(22) Filed: Mar. 18, 2004

(51) Int. Cl. A61F 2/06

(52) U.S. Cl. 623/1.23; 600/37

(57) ABSTRACT

A device used to hold and position a blood vessel in the performance of a coronary artery bypass graft procedure includes a handle and an attachment head coupled to the handle. The attachment head has a collar adapted to substantially encircle the blood vessel and having a number of suction apertures. A vacuum port is adapted to be coupled to a vacuum source and communicates a suction to the suction apertures to hold the blood vessel.

### FIELD OF THE INVENTION

The present invention relates to a device configured to atraumatically hold a blood vessel or other conduit during the performance of a surgical procedure. In particular, the present invention relates to a device that holds a graft vessel used to create an anastomosis during a coronary artery bypass graft procedure while presenting the open end of the graft in a configuration suitable for suturing at the anastomosis site.

### BACKGROUND OF THE INVENTION

A coronary artery bypass graft (CABG) procedure may be used in the treatment of coronary artery disease to bypass an obstruction in a coronary artery, thereby providing adequate blood flow to downstream heart tissues, lessening the risk of myocardial infarction.

Typical blood vessels used for the graft in a CABG procedure include an internal mammary artery (IMA), a greater saphenous vein (GSV) from the patient's leg, or a radial artery. The graft vessel is utilized as a shunt to avoid an obstruction, such as a bypass to the left anterior descending (LAD) coronary artery. Multiple bypass grafts may be utilized in a single surgery to bypass various obstructions.

In a CABG procedure, one end of a graft vessel is attached to a supply of arterial blood, and the other end is attached downstream of the obstruction in a coronary artery. The anastomosis may be a “T-graft” where the graft vessel is attached at a 90-degree angle to the side of another vessel or it may be a “Y-graft” where the end of the graft vessel is beveled to provide an angle between the graft vessel and the side of the vessel receiving the graft vessel, such as a 30 to 45-degree angle. Both T and Y grafts may be referred to as “end-to-side” anastomoses. During a CABG procedure, an “end-to-end” anastomosis may also be created where two vessel ends are attached to one another to create a longer conduit. Further, a lesser percentage of anastomoses may be “side-to-side” anastomoses where the sides of two vessels are connected.

Conventionally, an anastomosis is created by suturing the graft vessel to the anastomosis site. The graft vessel may be held apart from the anastomosis site while the sutures are put into place and then pulled up against the anastomosis site to complete the anastomosis. Typical ways of holding the graft vessel during the creation of the anastomosis include the use of a clip or clamp, a pair of forceps, or a robotic grasper.

CABG procedures may be performed using a cardiopulmonary bypass to provide a motionless heart. New procedures have become available to perform a CABG on a beating heart, which results in certain benefits to the patient when compared to placing the patient on cardiopulmonary bypass. Beating heart procedures may be performed utilizing a conventional open chest, such as through a median stemotomy to provide full access to the heart, but may also be performed utilizing an endoscopic procedure on a “closed” chest, using one or more small incisions in conjunction with an endoscope and specialized surgical instruments. When a CABG procedure is performed on a beating heart or without a stemotomy, it is referred to as minimally invasive cardiac surgery.

One challenge in performing a CABG procedure is holding and orienting the graft vessel when suturing the graft to the anastomosis site. Conventional holding mechanisms, such as a clip or forceps, or even an endoscopic robotic grasper in the case of minimally invasive cardiac surgery may not be specifically designed to hold the graft and therefore may be awkward to use, possibly lengthening the overall time of the CABG procedure. Further, ideally the open end of the graft should be held in an open position to facilitate suturing the graft to the anastomosis site, which may not be accomplished by conventional holding mechanisms, such as an alligator clip, that grasp the vessel. Further still, conventional holding mechanisms may not be designed to specifically avoid damaging the graft vessel. In particular, while the surgeon can manually use a delicate hold on the vessel when utilizing a forceps in an open heart procedure, a robotic grasper used in an endoscopic procedure may not provide direct feedback to the surgeon with respect to the force applied to the vessel when grasping, and therefore may damage the graft vessel.

In beating heart CABG procedures, new surgical instruments are being developed that aid in the creation of an anastomosis and are intended for insertion within the graft vessel during the procedure. For example, a balloon occlusion device may be utilized to occlude the aperture at the anastomosis site to prevent outflow of blood while the graft vessel is being attached. Such a balloon occlusion device may be inserted through a graft vessel and into the aorta, such as shown in U.S. Pat. No. 6,565,527 to Jonkman et al., issued May 20, 2003. Conventional holding instruments such as clips and forceps are not designed to hold the graft vessel securely with another surgical instrument inside the graft vessel because a clip or forceps will typically close off the lumen inside the vessel that is needed to accommodate the additional surgical instrument.

Accordingly, there is a need for an anastomosis device that is specifically designed to hold a graft vessel during the performance of a CABG to facilitate the procedure from a mechanical performance standpoint. Further, there is a need for a device that minimizes the time necessary to perform a CABG procedure by properly holding and positioning the graft vessel for suturing. Further, there is a need for a vessel-holding device that is designed to hold a graft vessel without damaging the vessel during a CABG procedure. Finally, it would be preferable to have a holding device that is configured to permit the insertion of another surgical instrument inside of the graft vessel while holding the graft.

It would be desirable to provide a system and/or method that provides one or more of these or other advantageous features. Other features and advantages will be made apparent from the present specification. The teachings disclosed extend to those embodiments that fall within the scope of the appended claims, regardless of whether they accomplish one or more of the aforementioned needs.

### SUMMARY OF THE INVENTION

One embodiment of the invention relates to a device used to hold and position a blood vessel in the performance of a coronary artery bypass graft procedure. The device has a handle and an attachment head coupled to the handle. The attachment head has a collar adapted to substantially encircle a blood vessel, the collar having a number of suction apertures. The device further has a vacuum port adapted to be coupled to a vacuum source, the vacuum port communicating a suction to the suction apertures to hold the blood vessel.

Another embodiment of the invention relates to a method of performing coronary artery bypass grafting surgery. The method includes the steps of creating an opening in a patient to access the heart of the patient, harvesting a blood vessel from the patient for use as a graft vessel, and providing a vessel holding and positioning device having a collar adapted to substantially encircle the blood vessel, the collar having a number of suction apertures adapted to engage the blood vessel. The method further includes the steps of providing a vacuum source coupled to the collar to create a suction at the suction apertures, inserting the blood vessel into the collar, and holding the blood vessel with the suction apertures. The method further includes the steps of positioning an end of the blood vessel with the vessel holding and positioning device at an anastomosis site on a second blood vessel, coupling the end of the blood vessel to the second blood vessel to create an anastomosis, removing the collar from the blood vessel, and closing the opening in the patient.

Another embodiment of the invention relates to a blood vessel positioning device for use in cardiac surgery. The blood vessel positioning device includes a handle, a first collar, and a second collar coupled to the handle. The second collar is separated from the first collar by a first distance. Each collar is adapted to substantially encircle a blood vessel and has a number of suction apertures, the suction apertures adapted to engage and hold the blood vessel.

Yet another embodiment of the invention relates to a method of creating an arteriotomy in a blood vessel. The method includes the steps of creating an opening in a patient to access the selected blood vessel in which to create the arteriotomy and providing a vessel holding and positioning device having at least two collars with a gap between one another, each collar adapted to substantially encircle the blood vessel, the collars each having a number of suction apertures adapted to engage the blood vessel. The method further includes the steps of providing a vacuum source coupled to the collars to create a suction at the suction apertures, inserting the blood vessel into the collars, holding the blood vessel with the suction apertures, an exposed portion of the blood vessel residing in the gap, and creating an arteriotomy in the exposed portion of the blood vessel.

The invention is capable of other embodiments and of being practiced or being carried out in various ways. Alternative exemplary embodiments relate to other features and combinations of features as may be generally recited in the claims.

### BRIEF DESCRIPTION OF THE DRAWINGS

The invention will become more fully understood from the following detailed description, taken in conjunction with the accompanying drawings, wherein like reference numerals refer to like elements, in which:

FIG. 1 is a schematic perspective view of a conduit holding and positioning system;

FIG. 2 is an exploded perspective view of the distal end of a conduit holding and positioning device;

FIG. 3 is a perspective view of the distal and of a conduit holding and positioning device shown holding a conduit;

FIG. 4 is a sectional view of the distal end of a conduit holding and positioning device shown holding a graft vessel proximate an anastomosis site;

FIG. 5 is a perspective view of the heart showing a conduit holding and positioning device holding a graft vessel at an anastomosis site in a coronary artery;

FIG. 6 is a perspective view of the distal end of a conduit holding and positioning device being removed from a graft vessel;

FIG. 7 is a perspective view of the distal end of a conduit holding and positioning device just after removal from a graft vessel;

FIG. 8 is a sectional view of the distal end of a conduit holding and positioning device shown holding a graft vessel and also receiving a surgical device;

FIG. 9 is a perspective view of a conduit holding and positioning device having multiple vacuum collars; and

FIG. 10 is an elevation view of the distal end of a conduit and positioning device having multiple vacuum collars being used to hold and position a conduit.

### DETAILED DESCRIPTION OF THE PREFERRED EMBODIMENTS

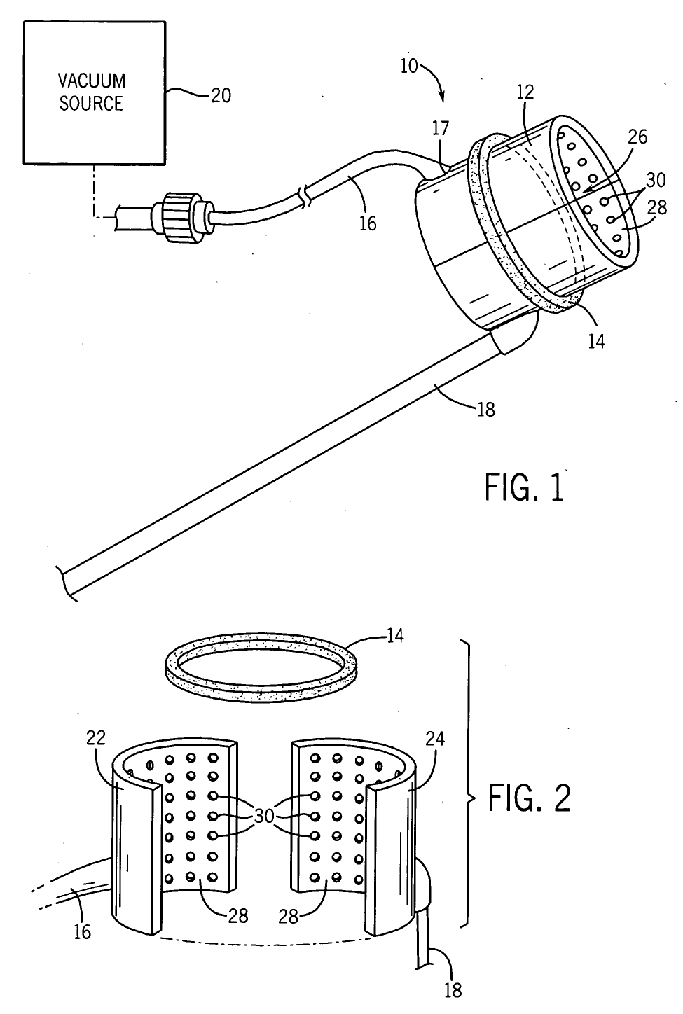

Referring to FIG. 1, according to an exemplary embodiment, a conduit holding and positioning device, shown as device 10, has a collar or cuff, shown as vacuum collar 12, and a handle, shown as positioning rod 18. A coupling mechanism or ring, shown as grasping ring 14, is disposed around vacuum collar 12. A vacuum line 16 extends from a vacuum port 17 that may be located directly on the vacuum collar 12. An interior lumen 26 having a lumen wall 28 extends through the vacuum collar 12. The lumen wall has a number of vacuum holes 30.

Further referring to FIG. 1, the vacuum line 16 may be coupled to a vacuum source 20. The vacuum source 20 may be a spring-loaded syringe sufficient to provide a suction at vacuum holes 30. In other embodiments, the vacuum source 20 may be an electrically powered vacuum pump or any other device known in the art that is able to provide a suction on vacuum line 16. While the vacuum line 16 is shown in FIG. 1 as a separate conduit extending from the vacuum collar 12, in other embodiments the vacuum line may be integrated into the handle, as shown in the embodiment depicted in FIG. 10. In such a case, the vacuum port 17 may reside at the connection point between the handle and the vacuum collar.

Further referring to FIG. 1, in an exemplary embodiment, the positioning rod 18 is constructed of surgical steel and includes a handle on one end to ease positioning by a surgeon. The rod 18 may be malleable such that a surgeon can bend the rod 18 into a particular orientation during surgery and have the rod 18 maintain that position. The rod 18 may have a device configured to allow the rod 18 to be attached to other surgical instruments, such as a retractor or a tissue stabilizer. The positioning rod 18 may be screwed into its attachment point on the vacuum collar 12 to allow it to be easily unscrewed. Other connection systems, such as a quick connect configuration, may also be used. A configuration that permits the positioning rod 18 to be easily connected and disconnected from the vacuum collar 12 permits a system in which various vacuum collars, such as vacuum collars of differing sizes, may be used with the same positioning rod 18.

Referring to FIG. 2, in an exemplary embodiment, vacuum collar 12 is made of a pair of vacuum collar halves 22, 24 that are held together in a cylindrical configuration by grasping ring 14. The vacuum holes 30 are interconnected with the vacuum line 16 such that the vacuum source 20 (see FIG. 1) creates a suction through the vacuum line 16 and consequently creates a suction at vacuum holes 30. While vacuum holes 30 are shown as a plurality of circular apertures, in other embodiments the vacuum holes may be other shapes such as ovals, diamonds, or rectangles. In still other embodiments, the vacuum holes 30 may be a series of spaced-apart rectangular slits or other channel-type configurations able to distribute the suction from vacuum source 20 around the lumen wall 28.

The two vacuum collar halves 22, 24 are shown as being held together with grasping ring 14. The grasping ring 14 may be constructed of a variety of materials sufficient to hold the two vacuum collar halves 22, 24 together during use of device 10. In other embodiments, other coupling mechanisms may be used to connect the vacuum collar halves 22, 24, such as snaps, latches, and other mechanical coupling mechanisms known in the art. Further, while the embodiment shown in FIG. 2 includes two vacuum collar halves, the vacuum collar 12 may be made of other numbers of pieces and still retain its functionality. In an exemplary embodiment, the vacuum collar 12 is made of an injectable plastic, but it may be made of other materials used in the art as well.

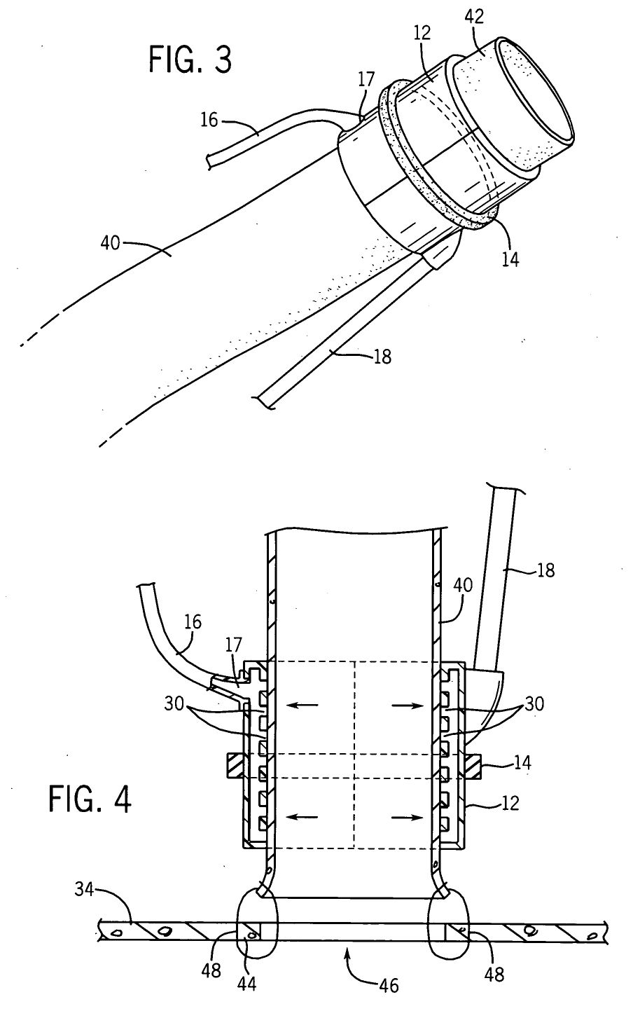

Referring to FIGS. 1 and 3, device 10 is configured to hold a conduit, such as a graft vessel 40, via the vacuum collar 12. When used in a CABG procedure, the graft vessel 40 may be a portion of the GSV or the left IMA. The graft vessel 40 is inserted into the vacuum collar 12 for holding purposes and is secured by the vacuum collar 12 when the vacuum source 20 provides a suction on the vacuum holes 30. The vacuum holes 30 suck the adventitia layer of the graft vessel 40 against the lumen wall 28 to hold the graft vessel 40 in place. In an exemplary embodiment, a distal end 42 of the graft vessel 40 is pulled through the vacuum collar 12, leaving the distal end 42 exposed.

The vacuum collar 12 may be of various sizes depending on the size of conduit that it is intended to hold. For example, an IMA is smaller in overall outer diameter than a GSV. The vacuum collar 12 may be available in various sizes between 1.5 millimeters and 5 millimeters in diameter in an exemplary embodiment. Other sizes may be applicable for use on other conduits within the body, such as a radial artery or a gastroepiploic artery. The grasping ring 14 is sized to properly hold the portions of the vacuum collar 12 together.

In a preferred embodiment, vacuum source 20 and vacuum holes 30 are configured such that the graft vessel 40 may be held securely and positioned by the surgeon without damaging the graft vessel 40. The vacuum holes 30 are distributed over the lumen wall 28 in a configuration that permits even pressure to be applied to many points on the graft vessel 40 to maintain position. Further, in a preferred embodiment, vacuum holes 30 are distributed on many points on lumen wall 28 so that graft vessel 40 is held in an open position to facilitate suturing of the distal end 42 and the possible insertion of additional surgical instruments through the graft vessel 40.

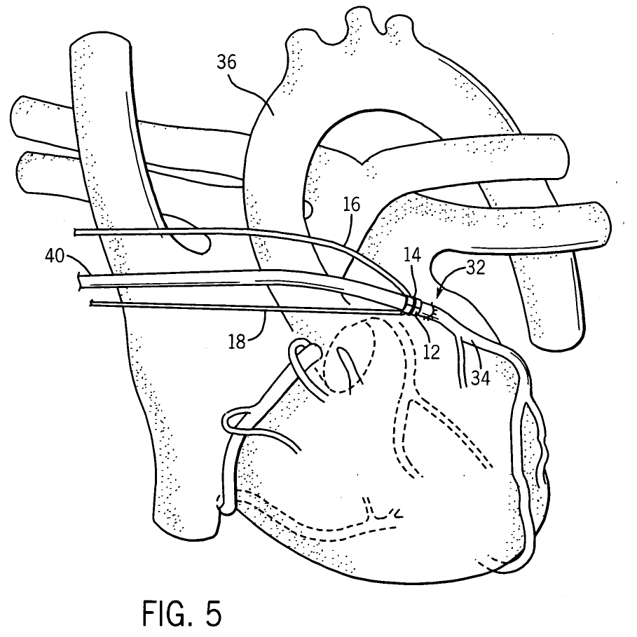

Referring to FIGS. 4 and 5, device 10 is shown holding graft vessel 40 proximate to an anastomosis site 32 in another blood vessel, shown as coronary artery 34. In an exemplary CABG procedure, graft vessel 40 is sutured to the vessel wall 44 of coronary artery 34 after an aperture 46 has been made in vessel wall 44. Commonly, sutures 48 are utilized to connect graft vessel 40 to vessel wall 44, and are first put into place while graft vessel 40 is held apart from but proximate to vessel wall 44 as depicted in FIG. 4 prior to tightening the sutures 48 to pull graft vessel 40 and vessel wall 44 together to create the anastomosis. The completed anastomosis is depicted in FIG. 5. A common coronary artery 34 is an LAD artery but may also be other coronary structures, such as the aorta 36. Further, rather than a coronary artery, other structures may be applicable in non-heart related surgical procedures, such as peripheral vascular or neurovascular procedures.

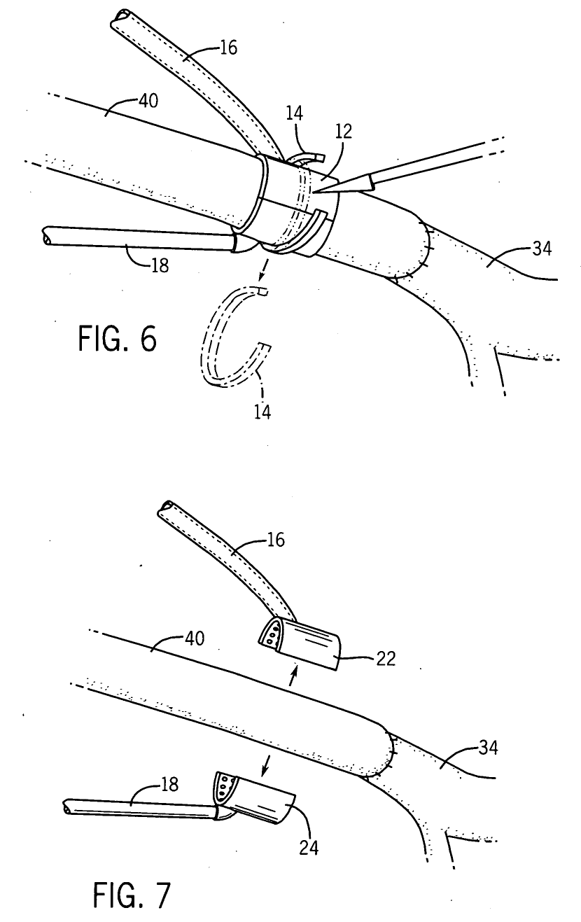

Referring to FIGS. 6 and 7, after graft vessel 40 has been attached to coronary artery 34 to create an anastomosis, the surgeon must remove the vacuum collar 12 from the graft vessel 40. In an exemplary embodiment, vacuum collar 12 is removed by cutting grasping ring 14, thereby removing grasping ring 14 and permitting the vacuum collar halves 22, 24 to be removed from graft vessel 40. Vacuum source 20 may be turned off or reversed prior to removing vacuum collar 12 to release the graft vessel 40 from the lumen wall 28. In other embodiments, the vacuum collar 12 may be removed in different ways depending on the type of connection between vacuum collar halves 22, 24 or the connection of other portions of the vacuum collar where vacuum collar halves 22, 24 are not utilized in the design.

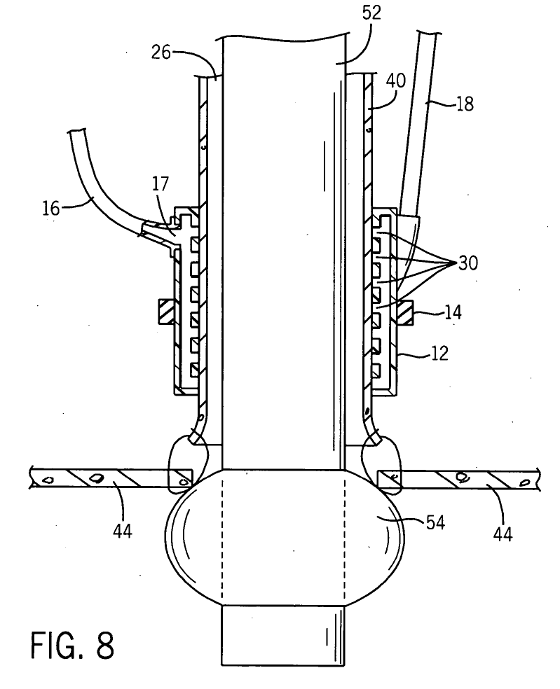

Referring to FIG. 8, vacuum collar 12 is structured to receive a surgical instrument, shown as, but not limited to, balloon catheter 52 within lumen 26. Balloon catheter 52 may be similar to the balloon catheter described in U.S. Pat. No. 6,565,527 to Jonkman et al., issued May 20, 2003. Balloon catheter 52 has a balloon 54 that is inflated after insertion into a blood vessel through vessel wall 44. Balloon 54 aids in performing a CABG procedure on a beating heart in circumstances where an anastomosis is created on a blood vessel that is actively carrying blood, such as when connecting one end of a GSV to the aorta to provide a blood supply. Other types of surgical instruments may also inserted into and received within vacuum collar 12 as well, such as for example a trocar, an aperture sealing system, or other type of endoscopic surgical instrument.

The conduit holding and positioning device is intended for use in beating heart CABG procedures and in procedures utilizing a heart lung machine. Further, the conduit holding and positioning device is contemplated for use in both endoscopic surgeries and open chest procedures. In an open chest procedure, a CABG begins with creating an opening in the chest, typically via a median sternotomy, and exposing the heart. A graft vessel is then harvested, such as a portion of the GSV from the patient's leg or one end of the left IMA from the patient's chest. When cardiopulmonary bypass is utilized, the patients circulatory system is attached to a heart lung machine and the heart is stopped. The harvested graft vessel may then be attached to the diseased coronary artery downstream of the obstruction utilizing the conduit holding and positioning device to aid in the procedure.

To use the conduit holding and positioning device, one end of the graft vessel is placed within the vacuum collar and the vacuum source is activated to secure the graft vessel within the vacuum collar. The handle is used by the surgeon to maneuver and position the graft vessel at the anastomosis site. Prior to attaching the graft vessel, an aperture is made in the diseased artery to accommodate the new blood supply from the graft vessel. The surgeon may create this aperture using a knife or. an endoscopic cutting instrument. The endoscopic instrument may be received within the conduit holding and positioning device. The graft vessel may then be sutured to the diseased artery to create the anastomosis. If the use of sutures is not required, such as in the case when using a non-suture based connection system, the connection system may be inserted through the conduit holding and positioning device, through the lumen of the graft vessel, and into the target artery to aid in or accomplish the anastomosis.

After the anastomosis has been created, the conduit holding and positioning device is removed from the graft vessel, such as by cutting the grasping ring and removing the vacuum collar. Another anastomosis may also be created depending on the number of bypasses required in the CABG operation to provide adequate blood flow to the diseased coronary arteries.

When utilizing a GSV rather than an IMA to provide a blood supply, the GSV must be connected to a blood supply, such as from the aorta. The conduit holding and positioning device may be used in a similar fashion to create an anastomosis to supply blood from the aorta to the GSV.

After the grafts have been completed, if cardiopulmonary bypass was used, the patient's heart is restarted and the circulatory system removed from the heart lung machine. The surgeon then closes the incision in the chest (either a large chest incision or the multiple ports used in endoscopic cardiac surgery) and completes the operation.

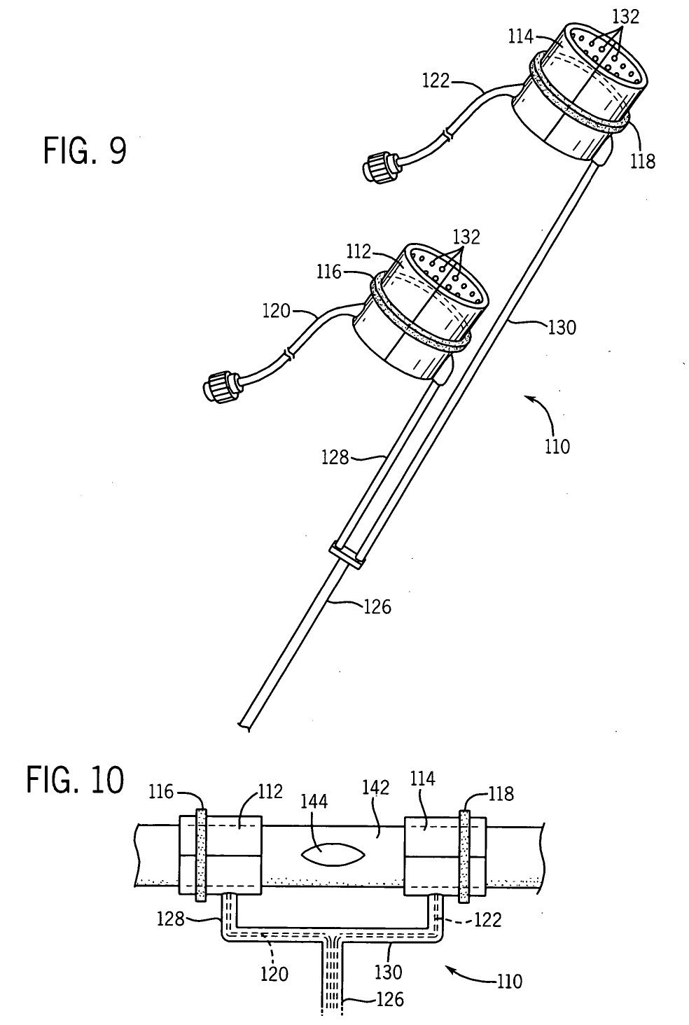

Referring to FIG. 9, according to another exemplary embodiment of the invention, conduit holding and positioning device 110 includes multiple collars, shown as vacuum collars 112, 114. Each of the vacuum collars 112, 114 includes a coupling mechanism, shown as grasping rings 116, 118 for vacuum collar removal purposes as described above. Each vacuum collar 112, 114 is provided with a vacuum line 120, 122 providing a vacuum source for internal suction apertures as described above with respect to holding and positioning device 10. The embodiment shown in FIG. 9 includes a handle 126 with a pair of rod branches 128, 130 extending to each vacuum collar 112, 114. The handle 126 and rod branches 128, 130 may be made of a malleable metal material to facilitate positioning of collars 112, 114. Alternatively, rod branches 128, 130 may be rigid to provide a consistent and fixed distance between the two vacuum collars 112, 114 for use in particular surgical procedures.

Further referring to FIG. 9, vacuum lines 120, 122 may be connected together to one vacuum source (not shown), but may have separate vacuum sources in different embodiments. Separate vacuum sources would allow the provision of a suction on one vacuum collar, without necessarily providing a suction on the other vacuum collar. As described above, vacuum collars 112, 114 function in a similar manner to vacuum collar 12 in that the vacuum source in conjunction with a number of vacuum holes 132 provides a method of atraumatically holding a conduit, such as a blood vessel.

Referring to FIG. 10, conduit holding and positioning device 110 is shown having a different handle configuration that may be useful in certain surgical procedures. Further, the vacuum lines 120, 122 are shown as incorporated into rod branches 128, 130 and handle 126, which may aid in the use of conduit holding and positioning device 110 by simplifying the overall mechanical structure of the device 110.

Further referring to FIG. 10, conduit holding and positioning device 110 is depicted as holding a blood vessel 142. In an exemplary embodiment, the blood vessel 142 is an IMA. The blood vessel 142 may be inserted through both vacuum collars 112, 114 by pulling the blood vessel 142 through the collars. Alternatively, the vacuum collars 112, 114 may have a hinged configuration that permit the collars 112, 114 to be opened, inserted over the target blood vessel, and closed or snapped together into the holding configuration depicted in FIG. 10.

Further referring to FIGS. 9 and 10, conduit holding and positioning device 110 may be used to aid in the creation of an arteriotomy 144, such as when a surgeon desires to create an anastomosis on an MA to provide a blood source to a second graft vessel used to supply blood to a diseased coronary artery (i.e. to create a branch line off the IMA). In such a circumstance, the vacuum collars 112, 114 may be fixed in place to provide a desired gap, such as 4 to 5 millimeters in one embodiment, to enable the creation of a precise arteriotomy 144. Further, the holding and positioning device 110 may be used to continue holding the blood vessel 142 during the creation of an anastomosis, thereby permitting control of both sides of the anastomosis site. Without the two vacuum collars 112, 114 holding and positioning the blood vessel 142, it can be challenging to grasp and position the blood vessel 142 to permit the precise incision in the blood vessel to create the arteriotomy 144 and the later creation of an anastomosis.

In an alternative embodiment, the device shown in FIGS. 9 and 10 may use one collar to occlude a blood vessel and the other collar to hold the blood vessel open. For example, vacuum collar 112 may apply pressure on the sides of vessel 142 (such as by providing a positive air pressure through holes 132) to close the interior flow channel. Stopping the blood flow through vessel 142 may be desired when the blood vessel is connected at one end to a source of blood and the other end is being used to create an anastomosis. Using the collar 112 to occlude the vessel may eliminate the need for a bulldog clamp or other occlusion device to accomplish that function. The other vacuum collar 114 may be used to open and position the end of the blood vessel 142 as described herein to expedite suturing or placement of an anastomotic device.

The conduit holding and positioning devices described in the various embodiments presented herein are intended to address conventional design challenges in providing a device that is able to atraumatically hold and position a conduit, such as a blood vessel. Further, when positioned near the end of a graft vessel, the conduit holding and positioning device may aid in presenting an open graft vessel distal end ideal for suturing at an anastomosis site. Further still, to the extent that the conduit holding and positioning device aids in the placement and maintaining the position of a graft vessel, it may reduce the mechanical complexity of an CABG procedure and permit the procedure to be performed in a shortened period of time. The conduit holding and positioning device may be especially helpful in endoscopic surgical procedures where surgical devices having a minimal “footprint” are desired for insertion through and use in small ports in the chest. Further, the ability to receive additional surgical instruments within the lumen of the conduit holding and positioning device may further aid in the performance of endoscopic and/or beating heat procedures. The vacuum concept may be especially important in reducing damage to graft vessels, especially as compared to robotic graspers used in endoscopic procedures that may not provide feedback to the surgeon with respect to the degree of force being placed on a blood vessel.

While the detailed drawings and specific examples given describe various exemplary embodiments, they serve the purpose of illustration only. It is to be understood that the invention is not limited in its application to the details of construction and the arrangements of components set forth in the preceding description or illustrated in the drawings. For example, the vacuum collars are, shown as cylindrical collars intended to encircle a conduit, such as a blood vessel. However, it is also contemplated that the vacuum collars may be only a portion of a cylinder but still able to hold and position a blood vessel by applying a vacuum source to a portion of the vessel wall, such as 180 degrees of a vessel wall and still perform the functions described herein. Further, the methods of use, such as surgical procedures, may be performed in a variety of sequences of steps. Furthermore, other substitutions, modifications, changes, and omissions may be made in the design, operating conditions, and arrangements of the exemplary embodiments without departing from the scope of the invention as expressed in the appended claims.

1. A device used to hold and position a blood vessel in the performance of a coronary artery bypass graft procedure, comprising:
a handle;
a collar coupled to the handle, the collar adapted to substantially encircle a blood vessel, the collar having a number of suction apertures; and
a vacuum port adapted to be coupled to a vacuum source, the vacuum port communicating a suction to the suction apertures to hold the blood vessel.

2. The device of claim 1, wherein the collar is comprised of two collar halves that together form a cylinder.

3. The device of claim 2, further comprising a ring encircling the collar halves to attach the collar halves together.

4. The device of claim 1, wherein the collar is sized to hold an internal mammary artery.

5. The device of claim 1, wherein the blood vessel is a graft vessel.

6. The device of claim 1, wherein the collar has a plurality of suction apertures and the suction apertures are circular.

7. The device of claim 1, wherein the handle is malleable.

8. The device of claim 1, further comprising a vacuum line adapted to couple the vacuum port to the vacuum source.

9. The device of claim 8, wherein the vacuum line is incorporated into the handle.

10. A conduit positioning system for use in the performance of a surgical technique on a patient, comprising:
a collar adapted to substantially encircle a conduit in the patient, the collar having a number of suction apertures;
a vacuum line coupled to the suction apertures;
a vacuum source coupled to the vacuum line to create a suction at the suction apertures to hold the conduit; and
a handle coupled to the attachment head.

11. The conduit positioning system of claim 10, wherein the collar is comprised of two collar halves that together form a cylinder.

12. The conduit positioning system of claim 11, further comprising a ring encircling the collar halves to attach the collar halves together.

13. The conduit positioning system of claim 10, wherein the collar is sized to hold an internal mammary artery.

14. The conduit positioning system of claim 10, wherein the conduit is a blood vessel.

15. The conduit positioning system of claim 10, wherein the collar has a plurality of suction apertures and the suction apertures are circular.

16. The conduit positioning system of claim 10, wherein the handle is malleable.

17. The conduit positioning system of claim 10, wherein the vacuum line is incorporated into the handle.

18. A method of performing coronary artery bypass grafting surgery, comprising:
creating an opening in a patient to access the heart of the patient;
harvesting a blood vessel from the patient for use as a graft vessel;
providing a vessel holding and positioning device, the vessel holding and positioning device having a collar adapted to substantially encircle the blood vessel, the collar having a number of suction apertures adapted to engage the blood vessel;
providing a vacuum source coupled to the collar to create a suction at the suction apertures;
inserting the blood vessel into the collar;
holding the blood vessel with the suction apertures;
positioning an end of the blood vessel with the vessel holding and positioning device at an anastomosis site on a second blood vessel;
coupling the end of the blood vessel to the second blood vessel to create an anastomosis;
removing the collar from the blood vessel; and
closing the opening in the patient.

19. The method of claim 18, wherein the blood vessel is an internal mammary artery or a greater saphenous vein.

20. The method of claim 19, wherein the collar comprises a plurality of wall portions coupled together with a ring and wherein the removing step comprises cutting the ring to remove the wall portions from around the blood vessel.

21. The method of claim 18, further comprising stopping the heart before creating the anastomosis.

22. The method of claim 18, wherein the opening is a median sternotomy, a mini-stemotomy, or a left anterior thoracotomy.

23. The method of claim 18, wherein the opening is an endoscopic port.

24. A blood vessel positioning device for use in cardiac surgery, comprising:
a handle; and
a first collar and a second collar coupled to the handle, the second collar separated from the first collar by a first distance, each collar adapted to substantially encircle a blood vessel and having a number of suction apertures, wherein the suction apertures are adapted to engage and hold the blood vessel.

25. The blood vessel positioning device of claim 24, wherein the handle comprises a first prong and a second prong, the first prong attached to the first collar and the second prong attached to the second collar.

26. The blood vessel positioning device of claim 24, wherein the first and second prongs are malleable whereby the distance may be changed.

27. The blood vessel positioning device of claim 24, wherein the collars are sized to encircle an internal mammary artery.

28. The blood vessel positioning device of claim 24, further comprising a vacuum source coupled to the suction apertures.

29. The blood vessel positioning device of claim 24, further comprising a vacuum line coupling the vacuum source to the first and second collars.

30. The blood vessel positioning device of claim 29, wherein the vacuum line is incorporated into the handle.

31. The blood vessel positioning device of claim 24, wherein each collar has a plurality of suction apertures and the suction apertures are circular.

32. A method of creating an arteriotomy in a blood vessel, comprising:
creating an opening in a patient to access a selected blood vessel in which to create the arteriotomy;
providing a vessel holding and positioning device, the vessel holding and positioning device having at least two collars with a gap between one another, each collar adapted to substantially encircle the blood vessel, the collars each having a number of suction apertures adapted to engage the blood vessel;
providing a vacuum source coupled to the collars to create a suction at the suction apertures;
inserting the blood vessel into the collars;
holding the blood vessel with the suction apertures, an exposed portion of the blood vessel residing in the gap; and
creating an arteriotomy in the exposed portion of the blood vessel.

33. The method of claim 32, wherein the vacuum source is coupled to the collars via a vacuum line incorporated into the handle.

34. The method of claim 33, wherein the blood vessel is an internal mammary artery.

35. The method of claim 32, wherein the opening is a median sternotomy, a mini-sternotomy, or a left anterior thoracotomy.

36. The method of claim 32, wherein the opening is an endoscopic port.

37. The method of claim 32, further comprising attaching a second blood vessel to the arteriotomy to create an anastomosis.
